SEQUENCE LISTING (3 sequences)

SEQ ID NO: 1 - DNA, 3767 bp, Severe Acute Respiratory Syndrome Coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacata

SEQ ID NO: 2 - DNA, 18 bp, Artificial Sequence
  Description of Artificial Sequence Synthetic primer
gcaagctgcagacgttgt

SEQ ID NO: 3 - DNA, 20 bp, Artificial Sequence
  Description of Artificial Sequence Synthetic primer
cagcaagaaccacaagagca

(19) United States
(12) Patent Application Publication
(10) Pub. No.: US 2005/0025788 A1
(43) Pub. Date: Feb. 3, 2005

(54) Systemic delivery of non-viral vector expressing SARS viral genomic vaccine

(76) Inventor: George Chin-Sheng Chou, Hsin-Shi (TW)

Correspondence Address: TROXELL LAW OFFICE PLLC, Suite 1404, 5205 Leesburg Pike, Falls Church, VA, 22041, US

(21) Appl. No.: 10/860,119

(22) Filed: Jun. 4, 2004

Related U.S. Application Data
(60) Provisional application No. 60/476,244, filed on Jun. 6, 2003.

(51) Int. Cl. A61K 39/215; A61K 39/12

(52) U.S. Cl. 424/221.1

(57) ABSTRACT

The present invention relates to a non-viral vector for SARS Viral Genomic Vaccine. The present invention also relates to a non-targeted lipoplex or PEGylated lipoplex formulation for accumulating SARS spike genome in the lung to that results in expression of SARS spike protein.

### DESCRIPTION OF PRIOR ART

Severe acute respiratory syndrome (SARS) is a respiratory illness that has been reported in Asia, North America, and Europe. In general, SARS begins with a fever greater than 100.4° F. (38.0° C.). Other symptoms may include headache, an overall feeling of discomfort, and body aches. Some people also experience mild respiratory symptoms. After 2 to 7 days, SARS patients may develop a dry cough and have trouble breathing.

Vaccination against a newly emerging SARS virus using inactivated virus or viral subunit component is the most effective approach for pandemic control of SARS virus. However, other options should be pursued according to the current knowledge and technology if antigenically-matched vaccines were not available in time or in sufficient quantity.

DNA vaccination has been reported to effectively elicit high-titer neutralizing antibody against influenza, measles, rabies, and herpes viruses. It can induce immune responses to epitopes that are highly conserved in viruses, while avoiding the risks of live-virus vaccines.

Lipids have been shown to be very effective agents for the delivery of nucleic acid into cells and there are numerous commercial reagents available for this purpose, including Lipofectin™ and LipofectAMINE™ (Gibco BRL). Plasmid transfection using such reagents is now a routine laboratory procedure commonly used in biomedical researches. Procedures for preparing liposomes for transfection formulations are described in U.S. Pat. Nos. 5,264,618 and 5,459,127, and by Felgner et al., Proc. Natl. Acad Sci. U.S.A. 84: 7413-7417, 1987.

For therapeutic applications that do not require sustained and regulated transgene expression, DNA-based immunization using non-viral gene delivery vehicle directly targeting muscle cells has become an attractive alternative to traditional immunization strategies. In addition to strong and long-lasting neutralizing antibody responses comparable with those seen in virally infected and convalescent animals that are elicited by single or singly boosted DNA immunizations, the mechanisms of DNA vaccination immunity most likely also include both T-cell immunity, in particular CD8<+> cytotoxic T-lymphocytes (CTL) and CD4<+> T cells, as well as antibodies against conserved epitopes.

### SUMMARY OF THE INVENTION

One embodiment of the present invention is a composition for DNA vaccination, comprising:

(i) a SARS spike protein genome in operative association with a non-viral vector; and

(ii) a vaccination vehicle comprising lipids.

Another embodiment of the invention is a process of eliciting immunity against SARS infection, comprising:

(i) DNA vaccination using the composition for DNA vaccination;

(ii) a subsequent local and/or systemic immunity against SARS spike protein.

### BRIEF DESCRIPTION OF THE DRAWINGS

FIG. 1 shows formulations used in SARS virus genomic vaccinations.

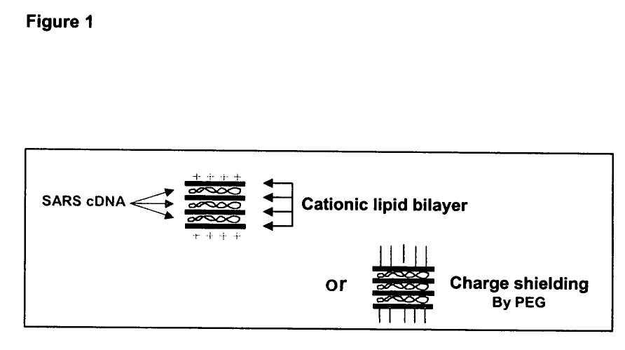

FIG. 2 shows strategies for systemic delivery of SARS protective genomes.

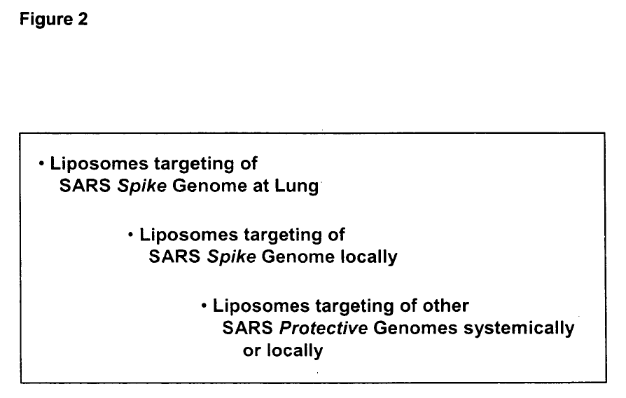

### DETAILED DESCRIPTION OF THE INVENTION

Systemically delivered non-modified cationic lipoplexes has been reported to control lung metastatic tumor and local tumor by James Mixson et al (James Mixson et al; Branched co-polymers of histidine and lysine are efficient carriers of plasmids, Nucleic Acid Research, 2001, Vol. 29, No. 6 1334-1340). In the present invention, non-targeted lipoplex or PEGylated lipoplex formulation aims at accumulation of SARS spike genome in the lung, which can express SARS spike proteins. A subsequent local and/or systemic immunity against SARS spike protein will be able to neutralize the infectivity of SARS viruses. A prophylactic effect would be beneficial to each vaccinee.

Various protective immunogens of SARS viruses can be delivered by non-viral systemic delivery vehicle. There is no payload restriction of transgene in non-viral vector formulation. The SARS viral immunogens is not manufactured but expressed in each vaccinee, therefore the invention may extensively decrease the demand of concurrent laborious work in manufacturing and purification of protein-based vaccine.

DNA vaccination is an effective means of eliciting strong humoral immunity to a number of viral antigens. DNA immunization expects to generate persistent, high-titer neutralizing antibody responses to human SARS viral spike protein (SP). Thus, systemically delivered DNA vaccine could provide adequate protection to this highly virulent pathogen. In comparison of the traditional glycoprotein-based vaccine, DNA vaccination with conserved SARS coronavirus genes provides a safe and useful first line protection that fights against a rapidly spreading pandemic virus by generating high-titer, persistent, neutralizing antibody with good avidity.

Accordingly, the present invention is provided with a composition for DNA vaccination, comprising:

(i) a SARS spike protein genome in operative association with a non-viral vector; and

(ii) a vaccination vehicle comprising lipids.

The non-viral vector expressing SARS spike protein genome is linked, either directly or through a linker, to the vaccination vehicle, wherein the vaccination vehicle is adapted to deliver the non-viral vector, thereby effecting expression of SARS spike genome.

Preparation

Polynucleotides:

The skilled person can readily construct a variety of clones containing functional nucleic acids. Cloning methodologies to accomplish these ends, and sequencing methods to verify the sequences of nucleic acids, are well known in the art. Examples of appropriate cloning and sequencing techniques, and instructions sufficient to direct persons of skill through many cloning exercises are found in Sambrook, et al., Molecular Cloning: A Laboratory Manual (2nd Ed., Vols. 1-3, Cold Spring Harbor Laboratory, 1989).

Product information from manufacturers of biological reagents and experimental equipment also provide information useful in known biological methods. Such manufacturers include the SIGMA chemical company (Saint Louis, Mo.), R & D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Polynucleotides containing the desired gene can be prepared by any suitable method including, for example, cloning and restriction of appropriate sequences as discussed supra, or by direct chemical synthesis by methods such as the solid support method of U.S. Pat. No. 4,458,066. Chemical synthesis produces a single stranded oligonucleotide. This may be converted into double stranded DNA by hybridization with a complementary sequence or by polymerization with a DNA polymerase using the single strand as a template. One of skill would recognize that while chemical synthesis of DNA is limited to sequences of about 100 bases, longer sequences might be obtained by the ligation of shorter sequences.

Nucleic acids may be modified by site-directed mutagenesis, as is well known in the art. Native and other nucleic acids can be amplified by in vitro methods. Amplification methods include the polymerase chain reaction (PCR), the ligase chain reaction (LCR), the transcription-based amplification system (TAS), the self-sustained sequence replication system (SSR). A wide variety of cloning methods, host cells, and in vitro amplification methodologies are well-known to persons of skill.

The Sequence of SARS-CoV Spike Protein:

| | |
|---|---|
| 21401 | |
| | ATGTTTATT |
| | TACAAATAA |
| | ApaLI |
| | ˜˜˜˜˜˜˜ |
| 21501 | TTCTTATTAT TTCTTACTCT CACTAGTGGT AGTGACCTTG ACCGGTGCAC |
| | CACTTTTGAT GATGTTCAAG CTCCTAATTA CACTCAACAT ACTTCATCTA |
| | AAGAATAATA AAGAATGAGA GTGATCACCA TCACTGGAAC TGGCCACGTG |
| | GTGAAAACTA CTACAAGTTC GAGGATTAAT GTGAGTTGTA TGAAGTAGAT |
| 21601 | TGAGGGGGGT TTACTATCCT GATGAAATTT TTAGATCAGA CACTCTTTAT |
| | TTAACTCAGG ATTTATTTCT TCCATTTTAT TCTAATGTTA CAGGGTTTCA |
| | ACTCCCCCCA AATGATAGGA CTACTTTAAA AATCTAGTCT GTGAGAAATA |
| | AATTGAGTCC TAAATAAAGA AGGTAAAATA AGATTACAAT GTCCCAAAGT |
| 21701 | TACTATTAAT CATACGTTTG GCAACCCTGT CATACCTTTT AAGGATGGTA |
| | TTTATTTTGC TGCCACAGAG AAATCAAATG TTGTCCGTGG TTGGGTTTTT |
| | ATGATAATTA GTATGCAAAC CGTTGGGACA GTATGGAAAA TTCCTACCAT |
| | AAATAAAACG ACGGTGTCTC TTTAGTTTAC AACAGGCACC AACCCAAAAA |
| 21801 | GGTTCTACCA TGAACAACAA GTCACAGTCG GTGATTATTA TTAACAATTC |
| | TACTAATGTT GTTATACGAG CATGTAACTT TGAATTGTGT GACAACCCTT |
| | CCAAGATGGT ACTTGTTGTT CAGTGTCAGC CACTAATAAT AATTGTTAAG |
| | ATGATTACAA CAATATGCTC GTACATTGAA ACTTAACACA CTGTTGGGAA |
| | NcoI |
| | ˜˜˜˜˜˜˜ |
| 21901 | TCTTTGCTGT TTCTAAACCC ATGGGTACAC AGACACATAC TATGATATTC |
| | GATAATGCAT TTAATTGCAC TTTCGAGTAC ATATCTGATG CCTTTTCGCT |
| | AGAAACGACA AAGATTTGGG TACCCATGTG TCTGTGTATG ATACTATAAG |
| | CTATTACGTA AATTAACGTG AAAGCTCATG TATAGACTAC GGAAAAGCGA |
| 22001 | TGATGTTTCA GAAAAGTCAG GTAATTTTAA ACACTTACGA GAGTTTGTGT |
| | TTAAAAATAA AGATGGGTTT CTCTATGTTT ATAAGGGCTA TCAACCTATA |
| | ACTACAAAGT CTTTTCAGTC CATTAAAATT TGTGAATGCT CTCAAACACA |
| | AATTTTTATT TCTACCCAAA GAGATACAAA TATTCCCGAT AGTTGGATAT |
| 22101 | GATGTAGTTC GTGATCTACC TTCTGGTTTT AACACTTTGA AACCTATTTT |
| | TAAGTTGCCT CTTGGTATTA ACATTACAAA TTTTAGAGCC ATTCTTACAG |
| | CTACATCAAG CACTAGATGG AAGACCAAAA TTGTGAAACT TTGGATAAAA |
| | ATTCAACGGA GAACCATAAT TGTAATGTTT AAAATCTCGG TAAGAATGTC |
| | PstI |
| | ˜˜˜˜˜˜˜ |
| 22201 | CCTTTTCACC TGCTCAAGAC ATTTGGGGCA CGTCAGCTGC AGCCTATTTT |
| | GTTGGCTATT TAAAGCCAAC TACATTTATG CTCAAGTATG ATGAAAATGG |
| | GGAAAAGTGG ACGAGTTCTG TAAACCCCGT GCAGTCGACG TCGGATAAAA |
| | CAACCGATAA ATTTCGGTTG ATGTAAATAC GAGTTCATAC TACTTTTACC |
| 22301 | TACAATCACA GATGCTGTTG ATTGTTCTCA AAATCCACTT GCTGAACTCA |
| | AATGCTCTGT TAAGAGCTTT GAGATTGACA AAGGAATTTA CCAGACCTCT |
| | ATGTTAGTGT CTACGACAAC TAACAAGAGT TTTAGGTGAA CGACTTGAGT |
| | TTACGAGACA ATTCTCGAAA CTCTAACTGT TTCCTTAAAT GGTCTGGAGA |
| 22401 | AATTTCAGGG TTGTTCCCTC AGGAGATGTT GTGAGATTCC CTAATATTAC |
| | AAACTTGTGT CCTTTTGGAG AGGTTTTTAA TGCTACTAAA TTCCCTTCTG |
| | TTAAAGTCCC AACAAGGGAG TCCTCTACAA CACTCTAAGG GATTATAATG |
| | TTTGAACACA GGAAAACCTC TCCAAAAATT ACGATGATTT AAGGGAAGAC |
| 22501 | TCTATGCATG GGAGAGAAAA AAAATTTCTA ATTGTGTTGC TGATTACTCT |
| | GTGCTCTACA ACTCAACATT TTTTTCAACC TTTAAGTGCT ATGGCGTTTC |
| | AGATACGTAC CCTCTCTTTT TTTTAAAGAT TAACACAACG ACTAATGAGA |
| | CACGAGATGT TGAGTTGTAA AAAAAGTTGG AAATTCACGA TACCGCAAAG |
| 22601 | TGCCACTAAG TTGAATGATC TTTGCTTCTC CAATGTCTAT GCAGATTCTT |
| | TTGTAGTCAA GGGAGATGAT GTAAGACAPA TAGCGCCAGG ACAAACTGGT |
| | ACGGTGATTC AACTTACTAG AAACGAAGAG GTTACAGATA CGTCTAAGAA |
| | AACATCAGTT CCCTCTACTA CATTCTGTTT ATCGCGGTCC TGTTTGACCA |
| 22701 | GTTATTGCTG ATTATAATTA TAAATTGCCA GATGATTTCA TGGGTTGTGT |
| | CCTTGCTTGG AATACTAGGA ACATTGATGC TACTTCAACT GGTAATTATA |
| | CAATAACGAC TAATATTAAT ATTTAACGGT CTACTAAAGT ACCCAACACA |
| | GGAACGAACC TTATGATCCT TGTAACTACG ATGAAGTTGA CCATTAATAT |
| | HindIII |
| | ˜˜˜˜˜˜˜ |
| 22801 | ATTATAAATA TAGGTATCTT AGACATGGCA AGCTTAGGCC CTTTGAGAGA |
| | GACATATCTA ATGTGCCTTT CTCCCCTGAT GGCAAACCTT GCACCCCACC |
| | TAATATTTAT ATCCATAGAA TCTGTACCGT TCGAATCCGG GAAACTCTCT |
| | CTGTATAGAT TACACGGAAA GAGGGGACTA CCGTTTGGAA CGTGGGGTGG |
| 22901 | TGCTCTTAAT TGTTATTGGC CATTAAATGA TTATGGTTTT TACACCACTA |
| | CTGGCATTGG CTACCAACCT TACAGAGTTG TAGTACTTTC TTTTGAACTT |
| | ACGAGAATTA ACAATAACCG GTAATTTACT AATACCAAAA ATGTGGTGAT |
| | GACCGTAACC GATGGTTGGA ATGTCTCAAC ATCATGAAAG AAAACTTGAA |
| 23001 | TTAAATGCAC CGGCCACGGT TTGTGGACCA AAATTATCCA CTGACCTTAT |
| | TAAGAACCAG TGTGTCAATT TTAATTTTAA TGGACTCACT GGTACTGGTG |
| | AATTTACGTG GCCGGTGCCA AACACCTGGT TTTAATAGGT GACTGGAATA |
| | ATTCTTGGTC ACACAGTTAA AATTAAAATT ACCTGAGTGA CCATGACCAC |
| 23101 | TGTTAACTCC TTCTTCAAAG AGATTTCAAC CATTTCAACA ATTTGGCCGT |
| | GATGTTTCTG ATTTCACTGA TTCCGTTCGA GATCCTAAAA CATCTGAAAT |
| | ACAATTGAGG AAGAAGTTTC TCTAAAGTTG GTAAAGTTGT TAAACCGGCA |
| | CTACAAAGAC TAAAGTGACT AAGGCAAGCT CTAGGATTTT GTAGACTTTA |
| 23201 | ATTAGACATT TCACCTTGCG CTTTTGGGGG TGTAAGTGTA ATTACACCTG |
| | GAACAAATGC TTCATCTGAA GTTGCTGTTC TATATCAAGA TGTTAACTGC |
| | TAATCTGTAA AGTGGAACGC GAAAACCCCC ACATTCACAT TAATGTGGAC |
| | CTTGTTTACG AAGTAGACTT CAACGACAAG ATATAGTTCT ACAATTGACG |
| 23301 | ACTGATGTTT CTACAGCAAT TCATGCAGAT CAACTCACAC CAGCTTGGCG |
| | CATATATTCT ACTGGAAACA ATGTATTCCA GACTCAAGCA GGCTGTCTTA |
| | TGACTACAAA GATGTCGTTA AGTACGTCTA GTTGAGTGTG GTCGAACCGC |
| | GTATATAAGA TGACCTTTGT TACATAAGGT CTGAGTTCGT CCGACAGAAT |
| 23401 | TAGGAGCTGA GCATGTCGAC ACTTCTTATG AGTGCGACAT TCCTATTGGA |
| | GCTGGCATTT GTGCTAGTTA CCATACAGTT TCTTTATTAC GTAGTACTAG |
| | ATCCTCGACT CGTACAGCTG TGAAGAATAC TCACGCTGTA AGGATAACCT |
| | CGACCGTAAA CACGATCAAT GGTATGTCAA AGAAATAATG CATCATGATC |
| 23501 | CCAAAAATCT ATTGTGGCTT ATACTATGTC TTTAGGTGCT GATAGTTCAA |
| | TTGCTTACTC TAATAACACC ATTGCTATAC CTACTAACTT TTCAATTAGC |
| | GGTTTTTAGA TAACACCGAA TATGATACAG AAATCCACGA CTATCAAGTT |
| | AACGAATGAG ATTATTGTGG TAACGATATG GATGATTGAA AAGTTAATCG |
| 23601 | ATTACTACAG AAGTAATGCC TGTTTCTATG GCTAAAACCT CCGTAGATTG |
| | TAATATGTAC ATCTGCGGAG ATTCTACTGA ATGTGCTAAT TTGCTTCTCC |
| | TAATGATGTC TTCATTACGG ACAAAGATAC CGATTTTGGA GGCATCTAAC |
| | ATTATACATG TAGACGCCTC TAAGATGACT TACACGATTA AACGAAGAGG |
| | ApaLI |
| | ˜˜˜˜˜˜ |
| 23701 | AATATGGTAG CTTTTGCACA CAACTAAATC GTGCACTCTC AGGTATTGCT |
| | GCTGAACAGG ATCGCAACAC ACGTGAAGTG TTCGCTCAAG TCAAACAAAT |
| | TTATACCATC GAAAACGTGT GTTGATTTAG CACGTGAGAG TCCATAACGA |
| | CGACTTGTCC TAGCGTTGTG TGCACTTCAC AAGCGAGTTC AGTTTGTTTA |
| 23801 | GTACAAAACC CCAACTTTGA AATATTTTGG TGGTTTTAAT TTTTCACAAA |
| | TATTACCTGA CCCTCTAAAG CCAACTAAGA GGTCTTTTAT TGAGGACTTG |
| | CATGTTTTGG GGTTGAAACT TTATAAAACC ACCAAAATTA AAAAGTGTTT |
| | ATAATGGACT GGGAGATTTC GGTTGATTCT CCAGAAAATA ACTCCTGAAC |
| 23901 | CTCTTTAATA AGGTGACACT CGCTGATGCT GGCTTCATGA AGCAATATGG |
| | CGAATGCCTA GGTGATATTA ATGCTAGAGA TCTCATTTGT GCGCAGAAGT |
| | GAGAAATTAT TCCACTGTGA GCGACTACGA CCGAAGTACT TCGTTATACC |
| | GCTTACGGAT CCACTATAAT TACGATCTCT AGAGTAAACA CGCGTCTTCA |
| 24001 | TCAATGGACT TACAGTGTTG CCACCTCTGC TCACTGATGA TATGATTGCT |
| | GCCTACACTG CTGCTCTAGT TAGTGGTACT GCCACTGCTG GATGGACATT |
| | AGTTACCTGA ATGTCACAAC GGTGGAGACG AGTGACTACT ATACTAACGA |
| | CGGATGTGAC GACGAGATCA ATCACCATGA CGGTGACGAC CTACCTGTAA |
| 24101 | TGGTGCTGGC GCTGCTCTTC AAATACCTTT TGCTATGCAA ATGGCATATA |
| | GGTTCAATGG CATTGGAGTT ACCCAAAATG TTCTCTATGA GAACCAAAAA |
| | ACCACGACCG CGACGAGAAG TTTATGGAAA ACGATACGTT TACCGTATAT |
| | CCAAGTTACC GTAACCTCAA TGGGTTTTAC AAGAGATACT CTTGGTTTTT |
| 24201 | CAAATCGCCA ACCAATTTAA CAAGGCGATT AGTCAAATTC AAGAATCACT |
| | TACAACAACA TCAACTGCAT TGGGCAAGCT GCAAGACGTT GTTAACCAGA |
| | GTTTAGCGGT TGGTTAAATT GTTCCGCTAA TCAGTTTAAG TTCTTAGTGA |
| | ATGTTGTTGT AGTTGACGTA ACCCGTTCGA CGTTCTGCAA CAATTGGTCT |
| 24301 | ATGCTCAAGC ATTAAACACA CTTGTTAAAC AACTTAGCTC TAATTTTGGT |
| | GCAATTTCAA GTGTGCTAAA TGATATCCTT TCGCGACTTG ATAAAGTCGA |
| | TACGAGTTCG TAATTTGTGT GAACAATTTG TTGAATCGAG ATTAAAACCA |
| | CGTTAAAGTT CACACGATTT ACTATAGGAA AGCGCTGAAC TATTTCAGCT |
| 24401 | GGCGGAGGTA CAAATTGACA GGTTAATTAC AGGCAGACTT CAAAGCCTTC |
| | AAACCTATGT AACACAACAA CTAATCAGGG CTGCTGAAAT CAGGGCTTCT |
| | CCGCCTCCAT GTTTAACTGT CCAATTAATG TCCGTCTGAA GTTTCGGAAG |
| | TTTGGATACA TTGTGTTGTT GATTAGTCCC GACGACTTTA GTCCCGAAGA |
| 24501 | GCTAATCTTG CTGCTACTAA AATGTCTGAG TGTGTTCTTG GACAATCAAA |
| | AAGAGTTGAC TTTTGTGGAA AGGGCTACCA CCTTATGTCC TTCCCACAAG |
| | CGATTAGAAC GACGATGATT TTACAGACTC ACACAAGAAC CTGTTAGTTT |
| | TTCTCAACTG AAAACACCTT TCCCGATGGT GGAATACAGG AAGGGTGTTC |
| 24601 | CAGCCCCGCA TGGTGTTGTC TTCCTACATG TCACGTATGT GCCATCCCAG |
| | GAGAGGAACT TCACCACAGC GCCAGCAATT TGTCATGAAG GCAAAGCATA |
| | GTCGGGGCGT ACCACAACAG AAGGATGTAC AGTGCATACA CGGTAGGGTC |
| | CTCTCCTTGA AGTGGTGTCG CGGTCGTTAA ACAGTACTTC CGTTTCGTAT |
| 24701 | CTTCCCTCGT GAAGGTGTTT TTGTGTTTAA TGGCACTTCT TGGTTTATTA |
| | CACAGAGGAA CTTCTTTTCT CCACAAATAA TTACTACAGA CAATACATTT |
| | GAAGGGAGCA CTTCCACAAA AACACAAATT ACCGTGAAGA ACCAAATAAT |
| | GTGTCTCCTT GAAGAAAAGA GGTGTTTATT AATGATGTCT GTTATGTAAA |
| 24801 | GTCTCAGGAA ATTGTGATGT CGTTATTGGC ATCATTAACA ACACAGTTTA |
| | TGATCCTCTG CAACCTGAGC TTGACTCATT CAAAGAAGAG CTGGACAAGT |
| | CAGAGTCCTT TAACACTACA GCAATAACCG TAGTAATTGT TGTGTCAAAT |
| | ACTAGGAGAC GTTGGACTCG AACTGAGTAA GTTTCTTCTC GACCTGTTCA |
| 24901 | ACTTCAAAAA TCATACATCA CCAGATGTTG ATCTTGGCGA CATTTCAGGC |
| | ATTAACGCTT CTGTCGTCAA CATTCAAAAA GAAATTGACC GCCTCAATGA |
| | TGAAGTTTTT AGTATGTAGT GGTCTACAAC TAGAACCGCT GTAAAGTCCG |
| | TAATTGCGAA GACAGCAGTT GTAAGTTTTT CTTTAACTGG CGGAGTTACT |
| 25001 | GGTCGCTAAA AATTTAAATG AATCACTCAT TGACCTTCAA GAATTGGGAA |
| | AATATGAGCA ATATATTAAA TGGCCTTGGT ATGTTTGGCT CGGCTTCATT |
| | CCAGCGATTT TTAAATTTAC TTAGTGAGTA ACTGGAAGTT CTTAACCCTT |
| | TTATACTCGT TATATAATTT ACCGGAACCA TACAAACCGA GCCGAAGTAA |
| 25101 | GCTGGACTAA TTGCCATCGT CATGGTTACA ATCTTGCTTT GTTGCATGAC |
| | TAGTTGTTGC AGTTGCCTCA AGGGTGCATG CTCTTGTGGT TCTTGCTGCA |
| | CGACCTGATT AACGGTAGCA GTACCAATGT TAGAACGAAA CAACGTACTG |
| | ATCAACAACG TCAACGGAGT TCCCACGTAC GAGAACACCA AGAACGACGT |
| 25201 | AGTTTGATGA GGATGACTCT GAGCCAGTTC TCAAGGGTGT CAAATTACAT |
| | TACACATA |
| | TCAAACTACT CCTACTGAGA CTCGGTCAAG AGTTCCCACA GTTTAATGTA |
| | ATGTGTAT |

Lipids:

Cationic lipids for use in the present invention include, for example, those described in U.S. Pat. Nos. 4,897,355, 5,264,618 and 5,459,127. Suitable lipids comprise, but are not limited to lysophosphatides, phosphatidylethanolamines, phosphatidylcholines, cholesterol derivatives, fatty acids, mono-, di- and tri-glyceride phospholipids having a neutral headgroup (Liu, et al., Nature Biotech. 15: 167-173, 1997; Hong,et al., FEBS Lett. 400:233-237, 1997). Other suitable single-chain lipids comprise the Rosenthal inhibitor ester and ether derivatives disclosed in U.S. Pat. Nos. 5,264,618 and 5,459,127.

Formation of Lipoplexes:

Lipid encapsulation is accomplished using liposomes that are able to stably bind or entrap and retain nucleic acid. The ratio of condensed DNA to lipid preparation can vary but will generally be around 1:1 (mg DNA:micromoles lipid), or more of lipid. Lipoplexes with a positive excess charge are typically used because they apparently interact better with the negatively charged surface of cells, and because cells can take them up better. For a review of the use of liposomes as carriers for delivery of nucleic acids, see, Hug and Sleight, Biochim. Biophys. Acta. 1097:1-17, 1991; Straubinger et al., in Methods of Enzymology, Vol. 101, pp. 512-527, 1983.

Liposomal preparations for use in the instant invention include cationic (positively charged), anionic (negatively charged) and neutral preparations, with cationic liposomes particularly preferred. Cationic liposomes have been shown to mediate intracellular delivery of plasmid DNA (Feigner et al., Proc. Natl. Acad. Sci. USA, 84:7413-7416, 1987); mRNA (Malone et al., Proc. Natl. Acad. Sci. USA, 86:6077-6081, 1989); and purified transcription factors (Debs et al., J. Biol. Chem., 265:10189-10192, 1990), in functional form.

Cationic Liposomes are Readily Available:

For example, N[1-2,3-dioleyloxy]propyl]-N,N,N-triethylammonium (DOTMA) liposomes are available under the trademark Lipofectin, from GIBCO BRL, Grand Island, N.Y. (See, also, Feigner et al., Proc. Natl. Acad. Sci. USA 84:7413-7416, 1987). Other commercially available lipids include transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA, 75:4194-4198, 1978; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others.

These materials can also be mixed with the DOTMA and DOTAP starting materials in appropriate ratios.

Methods for making liposomes using these materials are well known in the art.

The liposomes can comprise multilammelar vesicles (MLVs), small unilamellar vesicles (SUVs), or large unilamellar vesicles (LWs). The various liposome-nucleic acid complexes are prepared using methods known in the art such as Fraley et al., J. Biol. Chem., 255:10431, 1980; Szoka and Papahadjopoulos, Proc. Natl. Acad. Sci. USA, 75:145, 1978; and Schaefer-Ridder et al., Science, 215:166, 1982.

The DNA and/or protein antigen(s) can also be delivered in cochleate lipid compositions similar to those described by U.S. Pat. Nos. 4,663,161 and 4,871,488.

Linker:

According to embodiment of the present invention, if one or more of amino groups to which PEG chains bind, are reversibly protected by certain chemical groups from pegylation, the pegylation reaction will give directly the desired conjugate with specific pegylation sites, which can then be isolated from the reaction mixture, for example, by ultrafiltration or other chromatographic methods. In this case, the preparation method can further, optionally, comprise a de-protection reaction.

The “activated PEG” (or “pegylating agent”) is any PEG derivative, which can be used as protein modifier, because it contains a functional group capable of reacting with some functional croup in the protein/peptide to produce the PEG-protein/peptide conjugates. The activated PEG can be an alkylating reagent, such as PEG aldehyde, PEG epoxide or PEG tresylate, or it can be an acylating reagent, such as PEG ester.

Branched PEGs are also in common use. The branched PEGs can be represented as R(-PEG-OH)ₘ in which R represents a central core moiety such as pentaerythritol or glycerol, and m represents the number of branching arms. The number of branching arms (m) can range from three to a hundred or more. The hydroxyl groups are subject to chemical modification.

Subsequent SARS Immunity:

The present invention further provides a method of eliciting immunity against SARS infection, comprising:

(a) DNA vaccination using the composition of eliciting immunity against SARS infection, comprising: (i) SARS coronavirus spike protein genome in operative association with a non-viral vector; and (ii) vaccination vehicle comprising lipid; and

(b) subsequent local and/or systemic immunity against SARS spike protein.

In the present method, the subsequent local and/or systemic immunity against SARS is primarily by subcutaneously administration and then followed by intra-veneously boostering two weeks later.

### EXAMPLES

### Example 1

### Construction of SARS Spike Protein Plasmid (pCMVsp)

The pCMVsp plasmid is constructed by inserting SARS spike protein gene, the DNA sequence from 24,274 residue to 25,198 residue, (925 bps; see Attachment cDNAₛₚ) into pSecTag2 plasmid that contains an Igk chain secretion signal (Coloma, M J, et al, J. Imm. Methods, 1992; 152: 89-104.; Shiau J W, et al, Vaccine. 2000;19:1106-12; Locher C P, et al, DNA Cell Biol. 2002; 21: 581-6).
a. PCR
   i Materials
1. SARS spike protein cDNA (925 bps; Attachment cDNAₛₚ).
2. Primer for SARS spike protein genome:
   a. Sense primer: 5′-GCA AGC TGC AGA CGT TGT
   b. Antisense primer: 5′-CAG CAA GAA CCA CAA GAG CA
      ii. Methods
1. Amplify SARS spike protein genome by using SARS cDNA as the template.
2. Run agarose gel to check the size of PCR product.
b. Ligation
   i Materials
1. Vector—pSecTag2 plasmid (Invitrogen).
2. Ligase.
   ii Methods
1. Prepare the PCR product and ligate with vector—pSecTag2 using ligase.
2. Ligation at 16° C. overnight.
c. Transformation
   i Materials
1. Competent cells (JM109; Invitrogen).
2. LB growth medium.
   ii Methods
1. Gently mix the ligation mixtures with competent cells, and incubate the mixture on ice for 20 minutes.
2. Heat-shock the mixture at 42° C. for 90 sec and transfer to ice immediately.
3. Add 800 μl LB growth medium to the bacterial suspension. Shake in a 37° C. incubator for 45 minutes.
4. Plate 100 μl medium on a LB agar plate with ampicillin and culture at 37° C. for 12˜16 hours.
d. Checking
   i Methods
1. Pick a colony and seed into LB growth medium. Culture at 37° C. overnight.
2. Check the transformation efficiency by running on agarose gel and digesting with the corresponding restriction enzymes.

### Example 2

### Transfection

a. Materials

i. Lipofectamine 2000 (Invitrogen).
ii. Cos-7 cell (BCRC).
iii. Cell culture medium (Gibco BRL).
iv. Trypsin (Gibco BRL).
   b. Methods
i. The day before transfection, trypsinize and count the cells. Plate them in 24-well plates at 8×10⁴ cells per well so that they are 90-95% confluent on the day of transfection. Cells are plated in 0.5 ml of their normal growth medium containing serum and without antibiotics.
ii. For each well of cells to be transfected, dilute 0.8 μg of DNA into 50 μl of OPTI-MEM™ I Reduced Serum Medium without serum.
iii. For each well of cells, dilute 2.5 μl of LIPOFECTAMINE 2000 Reagent into 50 μl OPTI-MEM I Medium and incubate for 5 min at room temperature.
iv. Combine the diluted DNA with the diluted LF2000 reagent. Incubate at room temperature for 20 min to allow DNA-LF2000 Reagent complexes to form.
v. Add the DNA-LF2000 Reagent complexes (100 μl) directly to each well and mix gently by rocking the plate back and forth.
vi. Incubate the cells at 37° C. in a CO₂ incubator for a total of 24 h until they are ready to assay for transgene expression. It is not necessary to remove the complexes or change the medium. Alternatively, growth medium may be replaced after 4-6 h without loss in transfection activity.

### Example 3

### Western for Detecting Specific SARS Spike Protein Antigen

a. Materials

i. Specific SARS spike protein antigen from pCMVsp transfected cell culture supernatant.
ii. Polyclonal chicken anti-SARS spike protein IgY (from example 5).
iii. Goat anti-chicken IgY conjugated with HRP (2^{nd} Ab) (Promega).
iv. TMB substrate (Vector).
   b. Methods
i. Collect pCMVsp transfected cell culture supernatant.
ii. Boil the sample for 10 min.
iii. The samples are electrophoresed through SDS-PAGE and transferred to nitrocellulose membranes following standard procedures.
Iv. The western blot is blocked with blocking buffer and incubated with the plyclonal chicken anti-SARS spike protein IgY for 1 hr at RT.
v. The membrane is washed and subsequently incubated with goat anti-chicken IgY conjugated with HRP for 1 hr at room temperature.
vi. The membrane is washed again and the TMB substrate is added to develop color.
vii. Control of reaction.
viii. Positive Control: SARS spike protein antigen (from recombinant pCMVsp antigen of example 8).
ix. Negative Control: cell culture medium.

### Example 4

### Indirect Immunofluorescence Assay

a. Materials

i. Polyclonal chicken anti-SARS spike protein IgY (from example 5).
ii. Rabbit anti-chicken IgY conjugated with fluorescein isothiocyanate (FITC) (Promega).
iii Axioscope fluorescent microscope.
   b. Methods (Camenisch G, et al, FASEB J. 1999; 13(1): 81-88)
i. Cos-7 cells are seeded to silane coating slides and transfected with pCMVsp plasmid.
ii. After 24 hr post-transfection, cells are fixed with 4% paraformaldehyde or 100% methanol for 10 min at room temperature.
iii. Wash three times with PBS.
iv. The cells are permeabilized with 0.5% Triton X-100 in PBS for 5 min, and rinsed three times with PBS.
v. Add PBS containing 10% FCS for 30 min for blocking nonspecific binding.
vi. The cells are incubated with polyclonal chicken anti-SARS spike protein IgY at 37° C. for 1 hr.
vii. Wash with PBS.
viii. Add anti-chicken IgY conjugated with FITC at 37° C. for 1 hr.
ix. Wash with PBS and mount.
x. The result is detected by Axioscope microscope.

### Example 5

### Get Polyclonal Chicken Anti-SARS Spike Protein Antibody

a. Animal trial

i. Materials

1. Anti-SARS spike Ab negative hen.
2. pCMVsp plasmid.
3. Lipoplex.
   ii Methods
1. Immunize egg-generating hen with pCMVsp clone. Boost two times on the 2^{nd} and 4^{th} week after the first immunization.
2. If eggs were laid after the first immunization, collect the eggs.
3. Purify IgY from eggs using Promega's EGGstract IgY Purification System. The IgY will be used in ELISA (example 7) and indirect immunofluorescence assay (example 4).
   iii. IgY Purification
   i. Materials
1. EGGstract IgY Purification system (Promega).
   ii. Methods
1. Crack each egg and separate the egg white from the yolk using the egg separator.
2. Stir the yolk(s) at room temperature and slowly add 3 volumes of Precipitation Solution A.
3. Continue stirring the yolk mixtures for 5 min to precipitate the lipids; then centrifuge the mixture at 10,000×g at 4° C. for 10 min.
4. Collect the supernatant by filtering it through 4 layers of gauze.
5. Add ⅓ volume of Precipitation Solution B.
6. Continue stirring the yolk mixtures for 5 min to precipitate the IgY; then centrifuge the mixture at 10,000×g at 4° C. for 10 min.
7. Discard the supernatant. Resuspend the IgY pellet in a volume of PBS equal to the original volume of the egg yolk.
8. Repeat steps 5-7 to increase IgY purity to ˜90% and store at 4° C.
9. Analyze the IgY purity by SDS-PAGE. The concentration may be determined from the absorbance at 280 nm.

### Example 6

### DNA Vaccination and Bleeding

a. Materials

i. Rhode Island Red Chickens, 5-7 month old, Anti-SARS spike Ab negative.
ii. pCMVsp plasmid.
Iii Lipoplex.
   b. Methods
i. Anesthetize chicken and immunize chicken intravenously using 23-gauge needle. The immunization contains 500 μl lipoplexes with 2.5 μg or 5 μg pCMVsp DNA. Boost two times on the 2^{nd} and 4^{th} week after the first immunization (Song K D, et al, Vaccine. 2000; 19(2-3):2 43-52. Min W, et al, Vaccine. 2001; 20(1-2): 267-74.
ii. Collect blood before each immunization. After the 3^{rd} boosting, collect blood every two weeks until the 10^{th} week.

Iii groups:

10 chickens injected with 2.5 μg pCMVsp DNA/500 μl lipoplexes
10 chickens injected with 5 μg pCMVsp DNA/500 μl lipoplexes
6 chickens injected with 500 μl lipoplexes
6 chickens injected with 500 μl normal saline
Immunization time table.

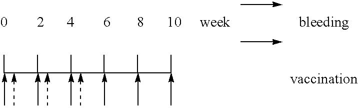

### Example 7

### ELISA for Detecting Anti-SARS Spike Protein IgG

a. Materials

i. Recombinant pCMVsp antigen.
ii. Serum of pCMVsp immunized chicken (anti-SARS spike Ab).
iii. Goat anti-chicken IgY conjugated with AP (2^{nd} Ab) (Promega).
iv Substrate-BCIP/NBT color development substrate (Promega).
v. ELISA reader.
vi. ELISA washer.
   b. Methods
i. Coating recombinant pCMVsp antigen on 96-well plate and incubate at 4° C. overnight.
ii. Rinse the plates 5 times with PBS-Tween washing solution. Add 1% BSA in PBS 200 μl per well of to block the plates. Incubate at room temperature for 30 minutes or overnight at 4° C.
iii. Add 50 μl serum of immunized chicken and incubate at 37° C. for 1 hr.
iv. Wash with PBS-Tween six times.
v. Add 100 μl anti-chicken IgY conjugated with AP and incubate at 37° C. for 1 hr.
vi. Wash with PBS-Tween six times.
vii. Add 100 μl of BCIP/NBT substrate to develop color and incubate at 37° C. for 1 hr.
viii. Read O.D. value by ELISA reader.
   c. Control of reaction
i. Positive Control: Polyclonal chicken anti-SARS spike protein antibody (Get Polyclonal chicken anti-SARS spike protein antibody from example 5).
ii. Negative Control: normal chicken sera.

### Example 8

### Get Recombinant pCMVsp Antigen

a. Construction of Recombinant Baculovirus

i. Materials

1. Baculovirus expression vector—pBlueBac4.5/V5-His (Invitrogen).
2. Competent cell (Invitrogen).
   ii. Methods
1. Digest pBlueBac4.5/V5-His and PCMVsp plasmid by restriction enzyme.
2. Ligate the SARS spike protein gene into pBlueBac4.5/V5-His.
3. Follow the transformation and checking procedures as example 1.
4. Collect the transformed clone as the transfer vector.
   b. Co-transfection
   i. Materials
1. Bac-N-Blue linear DNA (Invitrogen).
2. CellFECTIN™ reagent (Invitrogen).
3. Sf-9 insect cell (BCRC).
4. Cell culture medium (Grace's medium+FBS+supplement).
5. 28° C. incubator.
   ii. Methods
1. Transfer 1-2×10⁶ cells into a 35 mm tissue culture dish. The cells should be >95% viable. Allow cells to adhere for a few hours, or seed at a lower density and grow overnight.
2. Mix 1 μg of the Bac-N-Blue linearized viral vector with 4 μg of transfer vector, 1 ml media without serum, and 20 μl CellFECTIN reagent. Vortex for 10 seconds, then incubate at room temperature for 15 min.
3. Remove the media from the cells and wash with media without serum or antibiotics. Co-transfect these cells by slowly adding the liposome-DNA mixture in 0.5-1.0 ml media. Allow cells to incubate with this mixture for 5 hr. Adding 1.5 ml complete media to the transfected cells and incubate at 27° C. for about three days.
4. Wait until the cells show signs of infection—irregular shaped cells and increased volumes. Harvest the transfected cell supernatants after transfection.
   c. Plaque Assay
   i. Materials
1. 1% molten agarose.
2. X-gal.
   ii. Methods
1. Seed 35 mm tissue culture dishes with 1.5×10⁶ (or 1.0×10⁶) insect cells.
2. When cells have adhered and are at least 50% confluent, remove the medium and slowly add 200 μl of serially diluted supernatants from example 8-b-ii-4.
3. After 1 hour, aspirate the solution and overlay with 1.5 ml of pre-warmed 1% molten agarose (made by melting 2% SeaPlaque agarose in water, and then dilute with an equal volume of pre-warmed media).
4. Once the agarose has solidified, add 1.5 ml medium to each plate and transfer to a humidified storage box.
5. Bac-N-Blue linearized viral vector includes markers for identification of infected cells (ex. beta-galactosidase). Maintain the cells for 5-7 days. Aspirate the media, and stain cells with 0.03% X-gal diluted in medium.
6. Remove the X-gal solution after 5 hours, invert the plates and transfer them to a dark area. Check daily for blue plaques. Blue plaques typically appear after 5 days.
7. Infect additional insect cells with virus from these plaques to amplify the quantity and titer of viral stocks. Protein expression in these cells can be examined by Western Blot.
   d. Prepare cell lysate
   i. Methods
1. Harvest cells in 50 ml aliquots at the determined time point when cells have the maximal expression. Pellet cells by centrifugation and store at −70° C. until needed.
2. Resuspend cell pellet in 0.5X PBS containing 0.5 μg/ml Leupeptin (a protease inhibitor).
3. Lyse cells by applying two freeze-thaw cycles using a liquid nitrogen or dry ice/ethanol bath and a 42° C. water bath.
4. Shear DNA by passing the preparation through an 18-gauge needle four times.
5. Centrifuge the lysate at 3,000×g for 15 min to pellet the cellular debris. Transfer the supernatant to a fresh tube.
   e. Purification
   i. Materials
1. ProBond™ Purification System (Invitrogen).
   ii. Methods
1. Add the lysate to a prepared Purification Column (Invitrogen).
2. Let the supernatant bind to the column for 30-60 minutes under gentle agitation to keep the resin suspended in the lysate solution.
3. Settle the resin by gravity or low speed centrifugation (800×g), and carefully aspirate the supernatant. Save supernatant at 4° C. for SDS-PAGE analysis.
4. Wash columns with 8 ml Native Wash Buffer. Settle the resin by gravity or low speed centrifugation (800×g), and carefully aspirate the supernatant. Save supernatant at 4° C. for SDS-PAGE analysis.
5. Repeat Step 4 three more times.
6. Clamp the column in a vertical position and snap off the cap on the lower end. Elute the protein with 8-12 ml Native Elution Buffer. Collect 1 ml fractions and analyze with SDS-PAGE.
7. Get recombinant SARS spike protein antigen for ELISA assay.

### Example 9

### Preparation of Neutralizing Antibody

a. Materials

i. Vero cell (African green monkey kidney cells) (BCRC).
ii. Cell culture medium.
iii. Inactive SARS virus.
iv. nverted microscope.
   b. Methods
i. Seed 10⁵ vero cells/well in a 96-well tissue culture plate. Vero were maintained in Dulbecco's modified Eagle's minimal essential medium (DMEM, Gibco/BRL) with 10% FBS at 37° C. and 5% CO₂.
ii. Serially dilute serum from immunized chicken three times starting from 1:50.
iii. Add 100 TCID₅₀ vero cells into each diluted serum. Grow at 37° C., 5% CO₂ for 60 min.
iv. Add virus-antiserum mixtures into confluent cells, and grow for 72 hours.
v. Monitor the CPE. The antibody titer is measured as the highest dilution factor that does not result in CPE.

While the invention has been described with reference to a preferred embodiment thereof, it is to be understood that modifications or variations may be easily made without departing from the spirit of this invention, which is defined by the appended claims.

Attachment cDNA_{sp;} (The cDNA Sequence of SARS-CoV Spike Protein is indicated by Red Color.)

| | |
|---|---|
| 21401 | |
| | ATGTTTATT |
| | TACAAATAA |
| | ApaLI |
| | ˜˜˜˜˜˜˜ |
| 21501 | TTCTTATTAT TTCTTACTCT CACTAGTGGT AGTGACCTTG ACCGGTGCAC |
| | CACTTTTGAT GATGTTCAAG CTCCTAATTA CACTCAACAT ACTTCATCTA |
| | AAGAATAATA AAGAATGAGA GTGATCACCA TCACTGGAAC TGGCCACGTG |
| | GTGAAAACTA CTACAAGTTC GAGGATTAAT GTGAGTTGTA TGAAGTAGAT |
| 21601 | TGAGGGGGGT TTACTATCCT GATGAAATTT TTAGATCAGA CACTCTTTAT |
| | TTAACTCAGG ATTTATTTCT TCCATTTTAT TCTAATGTTA CAGCGTTTCA |
| | ACTCCCCCCA AATGATAGGA CTACTTTAAA AATCTACTCT GTGAGAAATA |
| | AATTGAGTCC TAAATAAAGA AGGTAAAATA AGATTACAAT GTCCCAAAGT |
| 21701 | TACTATTAAT CATACGTTTG GCAACCCTGT CATACCTTTT AAGGATGGTA |
| | TTTATTTTGC TGCCACAGAG AAATCAAATG TTGTCCGTGG TTGGGTTTTT |
| | ATGATAATTA GTATGCAAAC CGTTGGGACA GTATGGAAAA TTCCTACCAT |
| | AAATAAAACG ACGGTGTCTC TTTAGTTTAC AACAGGCACC AACCCAAAAA |
| 21801 | GGTTCTACCA TGAACAACAA GTCACAGTCG GTGATTATTA TTAACAATTC |
| | TACTAATGTT GTTATACGAG CATGTAACTT TGAATTGTGT GACAACCCTT |
| | CCAAGATGGT ACTTGTTGTT CAGTGTCAGC CACTAATAAT AATTGTTAAG |
| | ATGATTACAA CAATATGCTC GTACATTGAA ACTTAACACA CTGTTGGGAA |
| | NcoI |
| | ˜˜˜˜˜˜˜ |
| 21901 | TCTTTGCTGT TTCTAAACCC ATGGGTACAC AGACACATAC TATGATATTC |
| | GATAATGCAT TTAATTGCAC TTTCGAGTAC ATATCTGATG CCTTTTCGCT |
| | AGAAACGACA AAGATTTGGG TACCCATGTG TCTGTGTATG ATACTATAAG |
| | CTATTACGTA AATTAACGTG AAAGCTCATG TATAGACTAC GGAAAAGCGA |
| 22001 | TGATGTTTCA GAAAAGTCAG GTAATTTTAA ACACTTACGA GAGTTTGTGT |
| | TTAAAAATAA AGATGGGTTT CTCTATGTTT ATAAGGGCTA TCAACCTATA |
| | ACTACAAAGT CTTTTCAGTC CATTAAAATT TGTGAATGCT CTCAAACACA |
| | AATTTTTATT TCTACCCAAA GAGATACAAA TATTCCCGAT AGTTGGATAT |
| 22101 | GATGTAGTTC GTGATCTACC TTCTGGTTTT AACACTTTGA AACCTATTTT |
| | TAAGTTGCCT CTTGGTATTA ACATTACAAA TTTTAGAGCC ATTCTTACAG |
| | CTACATCAAG CACTAGATGG AAGACCAAAA TTGTGAAACT TTGGATAAAA |
| | ATTCAACGGA GAACCATAAT TGTAATGTTT AAAATCTCGG TAAGAATGTC |
| | PstI |
| | ˜˜˜˜˜˜˜ |
| 22201 | CCTTTTCACC TGCTCAACAC ATTTGGGGCA CGTCAGCTGC AGCCTATTTT |
| | GTTGGCTATT TAAACCCAAC TACATTTATG CTCAAGTATC ATCAAAATGG |
| | GGAAAAGTGG ACGAGTTCTG TAAACCCCGT CCAGTCGACC TCGCATAAAA |
| | CAACCGATAA ATTTCCGTTG ATGTAAATAC GAGTTCATAC TACTTTTACC |
| 22301 | TACAATCACA GATGCTGTTG ATTGTTCTCA AAATCCACTT GCTGAACTCA |
| | AATGCTCTGT TAAGAGCTTT GAGATTGACA AAGGAATTTA CCAGACCTCT |
| | ATGTTAGTGT CTACGACAAC TAACAAGAGT TTTAGGTGAA CGACTTGAGT |
| | TTACGAGACA ATTCTCGAAA CTCTAACTCT TTCCTTAAAT GGTCTGGAGA |
| 22401 | AATTTCAGGG TTGTTCCCTC AGCAGATCTT GTGAGATTCC CTAATATTAC |
| | AAACTTGTGT CCTTTTGGAG AGGTTTTTAA TGCTACTAAA TTCCCTTCTG |
| | TTAAAGTCCC AACAAGGGAG TCCTCTACAA CACTCTAAGG GATTATAATG |
| | TTTGAACACA GGAAAACCTC TCCAAAAATT ACCATGATTT AAGGGAAGAC |
| 22501 | TCTATGCATG GGAGAGAAAA AAAATTTCTA ATTGTGTTGC TGATTACTCT |
| | GTGCTCTACA ACTCAACATT TTTTTCAACC TTTAAGTGCT ATGGCGTTTC |
| | AGATACGTAC CCTCTCTTTT TTTTAAAGAT TAACACAACG ACTAATGAGA |
| | CACGAGATGT TGAGTTGTAA AAAAAGTTGG AAATTCACGA TACCGCAAAG |
| 22601 | TGCCACTAAG TTGAATGATC TTTGCTTCTC CAATGTCTAT GCAGATTCTT |
| | TTGTAGTCAA GGGAGATGAT GTAAGACAAA TAGCGCCAGG ACAAACTGGT |
| | ACGGTGATTC AACTTACTAG AAACGAAGAG GTTACAGATA CGTCTAAGAA |
| | AACATCAGTT CCCTCTACTA CATTCTGTTT ATCGCGGTCC TGTTTGACCA |
| 22701 | GTTATTGCTG ATTATAATTA TAAATTGCCA GATGATTTCA TGGGTTGTGT |
| | CCTTGCTTGG AATACTAGGA ACATTGATGC TACTTCAACT GGTAATTATA |
| | CAATAACGAC TAATATTAAT ATTTAACGGT CTACTAAAGT ACCCAACACA |
| | GGAACGAACC TTATCATCCT TGTAACTACG ATGAAGTTGA CCATTAATAT |
| | HindIII |
| | ˜˜˜˜˜˜˜ |
| 22801 | ATTATAAATA TAGGTATCTT AGACATGGCA AGCTTAGGCC CTTTGAGAGA |
| | GACATATCTA ATGTGCCTTT CTCCCCTGAT CGCAAACCTT GCACCCCACC |
| | TAATATTTAT ATCCATAGAA TCTGTACCGT TCGAATCCGG GAAACTCTCT |
| | CTGTATAGAT TACACGGAAA GACGGGACTA CCGTTTGGAA CCTGGGGTGG |
| 22901 | TGCTCTTAAT TGTTATTGGC CATTAAATGA TTATGGTTTT TACACCACTA |
| | CTGGCATTGG CTACCAACCT TACAGAGTTG TAGTACTTTC TTTTGAACTT |
| | ACGAGAATTA ACAATAACCG GTAATTTACT AATACCAAAA ATGTGGTGAT |
| | GACCGTAACC GATGGTTGGA ATGTCTCAAC ATCATGAAAG AAAACTTGAA |
| 23001 | TTAAATGCAC CGGCCACGGT TTGTGGACCA AAATTATCCA CTGACCTTAT |
| | TAAGAACCAG TGTGTCAATT TTAATTTTAA TGGACTCACT GGTACTGGTG |
| | AATTTACGTG GCCGGTGCCA AACACCTCGT TTTAATAGGT GACTGGAATA |
| | ATTCTTGGTC ACACAGTTAA AATTAAAATT ACCTGAGTGA CCATGACCAC |
| 23101 | TGTTAACTCC TTCTTCAAAG AGATTTCAAC CATTTCAACA ATTTGGCCGT |
| | GATGTTTCTG ATTTCACTGA TTCCGTTCGA GATCCTAAAA CATCTGAAAT |
| | ACAATTGAGG AAGAAGTTTC TCTAAAGTTG GTAAAGTTGT TAAACCGGCA |
| | CTACAAAGAC TAAAGTGACT AAGGCAAGCT CTAGGATTTT GTAGACTTTA |
| 23201 | ATTAGACATT TCACCTTGCG CTTTTGGGGG TGTAAGTGTA ATTACACCTG |
| | GAACAAATGC TTCATCTGAA GTTGCTGTTC TATATCAAGA TGTTAACTGC |
| | TAATCTGTAA AGTGGAACGC GAAAACCCCC ACATTCACAT TAATGTGGAC |
| | CTTGTTTACG AAGTAGACTT CAACGACAAC ATATAGTTCT ACAATTGACG |
| 23301 | ACTGATGTTT CTACAGCAAT TCATGCAGAT CAACTCACAC CAGCTTGGCG |
| | CATATATTCT ACTGGAAACA ATGTATTCCA GACTCAAGCA GGCTGTCTTA |
| | TGACTACAAA GATGTCGTTA AGTACGTCTA GTTGAGTGTG GTCGAACCGC |
| | GTATATAAGA TGACCTTTGT TACATAAGGT CTCAGTTCGT CCGACAGAAT |
| 23401 | TAGGAGCTGA GCATGTCGAC ACTTCTTATG AGTGCGACAT TCCTATTGGA |
| | GCTGGCATTT GTGCTAGTTA CCATACAGTT TCTTTATTAC GTACTACTAG |
| | ATCCTCGACT CGTACAGCTG TGAAGAATAC TCACGCTGTA AGGATAACCT |
| | CGACCGTAAA CACGATCAAT GGTATGTCAA AGAAATAATG CATCATGATC |
| 23501 | CCAAAAATCT ATTGTGGCTT ATACTATGTC TTTAGGTGCT GATAGTTCAA |
| | TTGCTTACTC TAATAACACC ATTGCTATAC CTACTAACTT TTCAATTAGC |
| | GGTTTTTAGA TAACACCGAA TATGATACAG AAATCCACGA CTATCAAGTT |
| | AACGAATGAG ATTATTGTGG TAACGATATG GATGATTGAA AAGTTAATCG |
| 23601 | ATTACTACAG AAGTAATGCC TGTTTCTATG GCTAAAACCT CCGTAGATTG |
| | TAATATGTAC ATCTGCGGAG ATTCTACTGA ATGTGCTAAT TTGCTTCTCC |
| | TAATGATGTC TTCATTACGG ACAAAGATAC CGATTTTGCA GGCATCTAAC |
| | ATTATACATG TAGACGCCTC TAAGATGACT TACACGATTA AACGAAGAGG |
| | ApaLI |
| | ˜˜˜˜˜˜ |
| 23701 | AATATGGTAG CTTTTGCACA CAACTAAATC GTGCACTCTC AGGTATTGCT |
| | GCTGAACAGG ATCGCAACAC ACGTGAAGTG TTCGCTCAAG TCAAACAAAT |
| | TTATACCATC GAAAACGTGT GTTGATTTAG CACGTGAGAG TCCATAACGA |
| | CGACTTGTCC TAGCGTTGTG TGCACTTCAC AAGCGAGTTC AGTTTGTTTA |
| 23801 | GTACAAAACC CCAACTTTGA AATATTTTGG TGGTTTTAAT TTTTCACAAA |
| | TATTACCTGA CCCTCTAAAC CCAACTAAGA GGTCTTTTAT TGAGGACTTG |
| | CATGTTTTGG GGTTGAAACT TTATAAAACC ACCAAAATTA AAAAGTGTTT |
| | ATAATGGACT GGGAGATTTC GGTTGATTCT CCAGAAAATA ACTCCTGAAC |
| 23901 | CTCTTTAATA AGGTGACACT CGCTGATGCT GGCTTCATGA AGCAATATGG |
| | CGAATGCCTA GGTGATATTA ATGCTAGAGA TCTCATTTGT GCGCAGAAGT |
| | GAGAAATTAT TCCACTGTGA GCGACTACGA CCGAAGTACT TCGTTATACC |
| | GCTTACGGAT CCACTATAAT TACGATCTCT AGAGTAAACA CGCGTCTTCA |
| 24001 | TCAATGGACT TACAGTGTTG CCACCTCTGC TCACTGATGA TATGATTGCT |
| | GCCTACACTG CTGCTCTAGT TAGTGGTACT GCCACTGCTG GATGGACATT |
| | AGTTACCTGA ATGTCACAAC GGTGGAGACG AGTGACTACT ATACTAACGA |
| | CGGATGTGAC GACGAGATCA ATCACCATGA CGGTGACGAC CTACCTGTAA |
| 24101 | TGGTGCTGGC GCTGCTCTTC AAATACCTTT TGCTATGCAA ATGGCATATA |
| | GGTTCAATGG CATTGGAGTT ACCCAAAATG TTCTCTATGA GAACCAAAAA |
| | ACCACGACCG CGACGAGAAG TTTATGGAAA ACGATACGTT TACCGTATAT |
| | CCAAGTTACC GTAACCTCAA TGGGTTTTAC AAGAGATACT CTTGGTTTTT |
| 24201 | CAAATCGCCA ACCAATTTAA CAAGGCGATT AGTCAAATTC AAGAATCACT |
| | TACAACAACA TCAACTGCAT TGGGCAAGCT GCAAGACGTT GTTAACCAGA |
| | GTTTAGCGGT TGGTTAAATT GTTCCGCTAA TCAGTTTAAG TTCTTAGTGA |
| | ATGTTGTTGT AGTTGACGTA ACCCGTTCGA CGTTCTGCAA CAATTGGTCT |
| 24301 | ATGCTCAAGC ATTAAACACA CTTGTTAAAC AACTTAGCTC TAATTTTGGT |
| | GCAATTTCAA GTGTGCTAAA TGATATCCTT TCGCGACTTG ATAAAGTCGA |
| | TACGAGTTCG TAATTTGTGT GAACAATTTG TTGAATCGAG ATTAAAACCA |
| | CGTTAAAGTT CACACGATTT ACTATAGGAA AGCGCTGAAC TATTTCAGCT |
| 24401 | GGCGGAGGTA CAAATTGACA GGTTAATTAC AGGCAGACTT CAAAGCCTTC |
| | AAACCTATGT AACACAACAA CTAATCAGGG CTGCTGAAAT CAGGCCTTCT |
| | CCGCCTCCAT GTTTAACTGT CCAATTAATG TCCGTCTGAA GTTTCGGAAG |
| | TTTGGATACA TTGTGTTGTT GATTAGTCCC GACGACTTTA GTCCCGAAGA |
| 24501 | GCTAATCTTG CTGCTACTAA AATGTCTGAG TGTGTTCTTG GACAATCAAA |
| | AAGAGTTGAC TTTTGTGGAA AGGGCTACCA CCTTATGTCC TTCCCACAAG |
| | CGATTAGAAC GACGATGATT TTACAGACTC ACACAAGAAC CTGTTAGTTT |
| | TTCTCAACTG AAAACACCTT TCCCGATGGT GGAATACAGG AAGGGTGTTC |
| 24601 | CAGCCCCGCA TGGTGTTGTC TTCCTACATG TCACGTATGT GCCATCCCAG |
| | GAGAGGAACT TCACCACAGC GCCAGCAATT TGTCATGAAG GCAAAGCATA |
| | GTCGGGGCGT ACCACAACAG AAGGATGTAC AGTGCATACA CGGTAGGGTC |
| | CTCTCCTTGA AGTGGTGTCG CGGTCGTTAA ACAGTACTTC CGTTTCGTAT |
| 24701 | CTTCCCTCGT GAAGGTGTTT TTGTGTTTAA TGGCACTTCT TGGTTTATTA |
| | CACAGAGGAA CTTCTTTTCT CCACAAATAA TTACTACAGA CAATACATTT |
| | GAAGGGAGCA CTTCCACAAA AACACAAATT ACCGTGAAGA ACCAAATAAT |
| | GTGTCTCCTT GAAGAAAAGA GGTGTTTATT AATGATGTCT GTTATGTAAA |
| 24801 | GTCTCAGGAA ATTGTGATGT CGTTATTGGC ATCATTAACA ACACAGTTTA |
| | TGATCCTCTG CAACCTGAGC TTGACTCATT CAAAGAAGAG CTGGACAAGT |
| | CAGAGTCCTT TAACACTACA GCAATAACCG TAGTAATTGT TGTGTCAAAT |
| | ACTAGGAGAC GTTGGACTCG AACTGAGTAA GTTTCTTCTC GACCTGTTCA |
| 24901 | ACTTCAAAAA TCATACATCA CCAGATGTTG ATCTTGGCGA CATTTCAGGC |
| | ATTAACGCTT CTGTCGTCAA CATTCAAAAA GAAATTGACC GCCTCAATGA |
| | TGAAGTTTTT AGTATGTAGT GGTCTACAAC TAGAACCGCT GTAAAGTCCG |
| | TAATTGCGAA GACAGCAGTT GTAAGTTTTT CTTTAACTGG CGGAGTTACT |
| 25001 | GGTCGCTAAA AATTTAAATG AATCACTCAT TGACCTTCAA GAATTGGGAA |
| | AATATGAGCA ATATATTAAA TGGCCTTGGT ATGTTTGGCT CGGCTTCATT |
| | CCAGCGATTT TTAAATTTAC TTAGTGAGTA ACTGGAAGTT CTTAACCCTT |
| | TTATACTCGT TATATAATTT ACCGGAACCA TACAAACCGA GCCGAAGTAA |
| 25101 | GCTGGACTAA TTGCCATCGT CATGGTTACA ATCTTGCTTT GTTGCATGAC |
| | TAGTTGTTGC AGTTGCCTCA AGGGTGCATG CTCTTGTGGT TCTTGCTGCA |
| | CGACCTGATT AACGGTAGCA GTACCAATGT TAGAACGAAA CAACGTACTG |
| | ATCAACAACG TCAACGGAGT TCCCACGTAC GAGAACACCA AGAACGACGT |
| 25201 | AGTTTGATGA GGATGACTCT GAGCCAGTTC TCAAGGGTGT CAAATTACAT |
| | TACACATA |
| | TCAAACTACT CCTACTGAGA CTCGGTCAAG AGTTCCCACA GTTTAATGTA |
| | ATGTGTAT |

1. A composition of eliciting immunity against SARS infection, comprising:
(i) SARS coronavirus spike protein genome in operative association with a non-viral vector; and
(ii) vaccination vehicle comprising lipid.

2. The composition of claim 1, wherein the vector containing the SARS coronavirus protein genome is linked directly or through a linker to the vaccination vehicle that comprises lipid, wherein the vaccination vehicle is adapted to deliver the vector into the target cell.

3. The composition of claim 1, wherein the genome sequence of SARS coronavirus spike protein is shown in Attachment cDNAₛₚ.

4. The composition of claim 1, wherein the lipid is selected from the group consisting of cationic lipids, anionic and neutral liposomes and lipoplexes,

5. The composition of claim 4, wherein the lipid is lipoplexes.

6. The composition of claim 2, wherein the linker is selected from the group consisting of activated PEG, branched PEG and PEG.

7. The composition of claim 6, wherein the linker is PEG.

8. A method of eliciting immunity against SARS infection, comprising:
(a) DNA vaccination using the composition of composition of eliciting immunity against SARS infection, comprising: (i) SARS coronavirus spike protein genome in operative association with a non-viral vector; and (ii) vaccination vehicle comprising lipid; and
(b) subsequent local and/or systemic immunity against SARS spike protein.

9. The method according to claim 8, wherein the subsequent local and/or systemic immunity against SARS is by i.v. administration.

10. The method of claim 8, wherein the vector containing the SARS coronavirus protein genome is linked directly or through a linker to the vaccination vehicle comprising lipid, wherein the vaccination vehicle is adapted to deliver the vector into the target cell.

11. The method of claim 8, wherein the genome sequence of SARS coronavirus spike protein is shown in Attachment cDNAₛₚ.

12. The method of claim 8, wherein the lipid is selected from the group consisting of cationic lipids, anionic and neutral liposomes and lipoplexes,

13. The method of claim 12, wherein the lipid is lipoplexes.

14. The method of claim 10, wherein the linker is selected from the group consisting of activated PEG, branched PEG, and PEG.

15. The method of claim 14, wherein the linker is PEG.
